(19) United States
(12) Patent Application Publication
(10) Pub. No.: US 2006/0224244 A1
(43) Pub. Date: Oct. 5, 2006

(54) Hydrogel implant

(75) Inventors: Brian H. Thomas, Columbia City, IN (US); Donald Yakimicki, Plymouth, IN (US); Steven J. Charlebois, Goshen, IN (US); Michael E. Hawkins, Columbia City, IN (US)

Correspondence Address: ZIMMER TECHNOLOGY - REEVES, P. O. BOX 1268, ALEDO, TX, 76008, US

(73) Assignee: Zimmer Technology, Inc.

(21) Appl. No.: 11/095,217

(22) Filed: Mar. 31, 2005

(51) Int. Cl. A61F 2/38; A61F 2/30

(52) U.S. Cl. 623/20.28; 623/18.11; 623/23.5; 623/23.51; 264/425

(57) ABSTRACT

A hydrogel implant for replacing a portion of a skeletal joint.

### FIELD OF THE INVENTION

The invention relates to implants for skeletal joints. In particular, the invention relates to such implants having hydrogel bearing surfaces.

### BACKGROUND

Degenerative and traumatic damage to the articular cartilage of skeletal joints can result in pain and restricted motion. Prosthetic joint replacement surgery is frequently utilized to alleviate the pain and restore joint function. During this surgery, one or more of the articulating surfaces of the joint are replaced with prosthetic bearing components. The replacement components typically include a portion for anchoring the implant adjacent to the joint and a portion for articulating with opposing joint surfaces. It is desirable for the implant to be well anchored and present a low friction, low wear articular surface.

Modular joint implants have become popular because they allow the surgeon to assemble components in a variety of configurations at the time of surgery to meet specific patient needs relative to fit and function. For example, modular implants may include separate anchorage and articular components that can be assembled in a variety of configurations of surface finish, fixation mechanism, size, kinematic constraint, and/or other parameters to suit a particular patient's condition. Where such modular components are supplied, a means for attaching them to one another is typically provided.

### SUMMARY

The present invention provides a hydrogel implant for replacing a portion of a skeletal joint.

In one aspect of the invention, an implant for replacing a portion of a skeletal joint includes an articular surface comprising a hydrogel and a porous substrate. The hydrogel is attached to the substrate by interdigitation of a portion of the hydrogel into some of the pores of the substrate.

In another aspect of the invention, an implant for replacing a portion of a skeletal joint includes an articular surface comprising a hydrogel, a substrate, a modular base plate, and a locking mechanism. The hydrogel is attached to a first portion of the substrate and a second portion of the substrate forms an engagement portion. The engagement portion of the substrate is engageable with the base plate and the locking mechanism locks the substrate in engagement with the base plate.

In another aspect of the invention, an implant for replacing a portion of a skeletal joint includes a hydrogel articular surface and an integral substrate for supporting the hydrogel. The substrate is more highly crosslinked than the articular surface.

In another aspect of the invention, a method of forming an implant for replacing a portion of a skeletal joint includes forming an implant having a hydrogel articular surface and a substrate; and irradiating the implant adjacent to the substrate.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is an exploded perspective view of an implant according to the present invention;

FIG. 2 is a bottom view of one component of the implant of FIG. 1; and

FIG. 3 is a cross sectional view of the component of FIG. 2 taken along line 3-3.

### DESCRIPTION OF THE ILLUSTRATIVE EXAMPLES

Embodiments of a hydrogel implant include a hydrogel bearing mounted to a substrate. The hydrogel implant may function as a replacement for damaged or diseased cartilage of a skeletal joint to sustain continued joint function. The hydrogel implant may be used to replace a portion of any skeletal joint including, but not limited to, joints of the hip, knee, shoulder, spine, elbow, wrist, ankle, jaw, and digits. The implant may be configured to replace a relatively small defect within the joint, an entire compartment of the joint, and/or the total joint.

The hydrogel bearing includes a three dimensional network of polymer chains with water filling the void space between the macromolecules. The hydrogel includes a water soluble polymer that is crosslinked to prevent its dissolution in water. The water content of the hydrogel may range from 20-80%. The high water content of the hydrogel results in a low coefficient of friction for the bearing due to hydrodynamic lubrication. Advantageously, as loads increase on the bearing component, the friction coefficient decreases as water forced from the hydrogel forms a lubricating film. The hydrogel may include natural or synthetic polymers. Examples of natural polymers include polyhyaluronic acid, alginate, polypeptide, collagen, elastin, polylactic acid, polyglycolic acid, chitin, and/or other suitable natural polymers and combinations thereof. Examples of synthetic polymers include polyethylene oxide, polyethylene glycol, polyvinyl alcohol, polyacrylic acid, polyacrylamide, poly(N-vinyl-2-pyrrolidone), polyurethane, polyacrylonitrile, and/or other suitable synthetic polymers and combinations thereof. For example, the hydrogel may include a crosslinked blend of polyvinyl alcohol (PVA) and poly(N-vinyl-2-pyrrolidone) (PVP). The hydrogel may also include beneficial additives that are released at the surgical site. For example, the hydrogel may include analgesics, antibiotics, growth factors, and/or other suitable additives.

The substrate provides support for the hydrogel and/or provides an anchor for the implant. The substrate may include an open porous structure in which the hydrogel is integrated to attach the hydrogel to the substrate. The substrate may include an open porous structure for placement adjacent to body tissue to receive tissue ingrowth to anchor the implant adjacent the tissue. The porous structure may be configured to promote hard and/or soft tissue ingrowth. The porous structures may be in form of an open cell foam, a woven structure, a grid, agglomerated particles, and/or other suitable structures and combinations thereof. Alternatively, the substrate may engage a separate modular base plate that forms the anchoring portion of the implant. The implant may include a locking mechanism for locking the substrate in engagement with the base plate. The locking mechanism may include interlocking dovetails, clips, springs, screws, bolts, pins, and/or other locking mechanisms.

The substrate may include any suitable material including, but not limited to, metals, polymers, ceramics, hydrogels and/or other suitable materials and combinations thereof. For example, a metal substrate may include titanium, tantalum, stainless steel, and/or other suitable metals and alloys thereof. A polymer substrate may include resorbable and/or non-resorbable polymers. Examples of resorbable polymers include polylactic acid polymers, polyglycolic acid polymers, and/or other suitable resorbable polymers. Examples of non-resorbable polymers include polyolefins, polyesters, polyimides, polyamides, polyacrylates, polyketones, and/or other suitable non-resorbable polymers. For example, the substrate may include a foamed, porous polyethylene body having a first surface to which the hydrogel is attached and a second surface engageable with an optional base plate.

The hydrogel may be formed by solution casting, injection molding, compression molding, and/or other suitable forming processes. The hydrogel may be crosslinked using freeze thaw cycling, gamma ray irradiation, ultraviolet irradiation, electron beam irradiation, chemical crosslinking agents, and/or other suitable crosslinking methods. The hydrogel may be formed directly onto a porous substrate such that the hydrogel interdigitates with a portion of the substrate. The hydrogel may be further crosslinked, such as by irradiation, after forming onto a porous substrate to strengthen the portion of the hydrogel interdigitated into the substrate. If the substrate includes an organic substance or is modified to have organic groups at its surface, irradiation of the hydrogel-to-substrate interface will result in crosslinking of the hydrogel to the substrate such that the resulting covalent bonds will increase the hydrogel-to-substrate bond strength.

The hydrogel may be formed with an integral substrate by relatively highly crosslinking a portion of the hydrogel to form a strong substrate portion and relatively lightly crosslinking a different portion of the hydrogel to form a bearing surface.

The hydrogel implant may further include an opposing joint component for articulation with the hydrogel bearing.

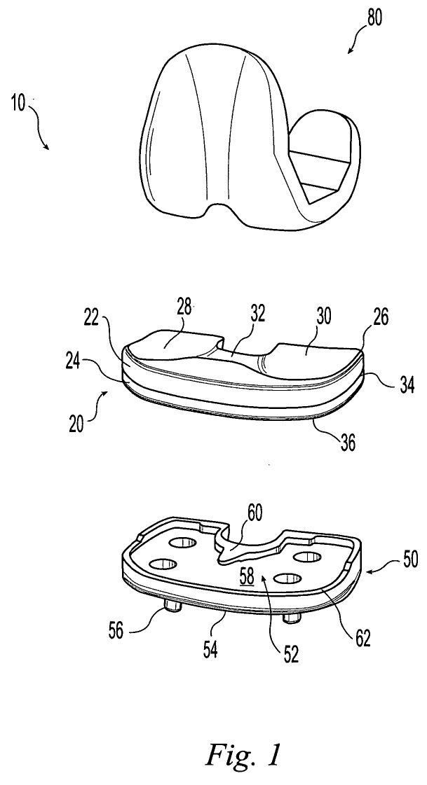

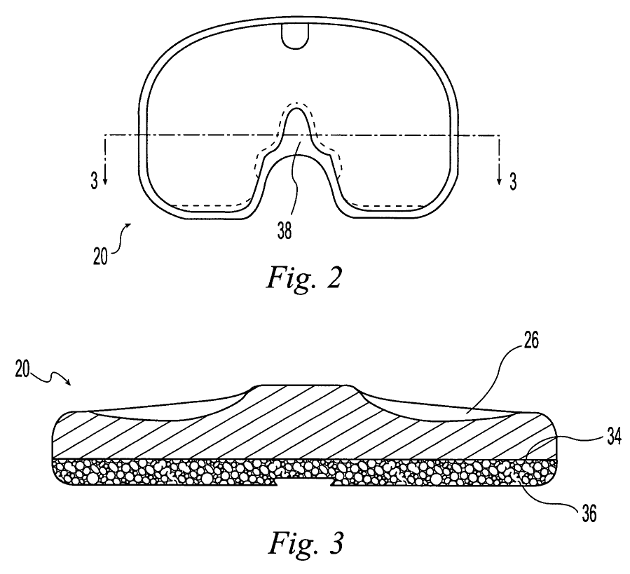

FIGS. 1-3 depict an illustrative example of an implant 10 according to the present invention. The illustrative implant 10 is in the form of a knee joint prosthesis and includes a hydrogel implant 20 and an optional tibial base plate 50 and an optional femoral implant 80. The illustrative hydrogel implant 20 is configured to replace the entire articular bearing surface of a tibia at a knee joint and to articulate with the femoral condyles or optionally with the prosthetic femoral implant 80. However, it is within the scope of the invention for the hydrogel implant 20 to be configured to replace only a portion of the articular bearing surface, to replace the femoral condyles of the knee joint, and/or to replace any amount of any bearing surface in any skeletal joint.

The hydrogel implant 20 includes a hydrogel bearing 22 mounted to a substrate 24. The bearing 22 includes a bearing surface 26 configured to receive an opposing portion of the joint. In the illustrative example, the bearing surface 26 includes medial and lateral articular regions 28, 30 separated by an intercondylar portion 32. The substrate 24 preferably includes a first porous region 34 in which the bearing 22 is interdigitated to connect the bearing 22 to the substrate 24. In the illustrative example, a crosslinked hydrogel is compression molded into the first porous region 34. The hydrogel may be subsequently further crosslinked to strengthen the interdigitating portion and lock the hydrogel to the substrate. For example, any of the above listed hydrogels and mixtures of them will crosslink when irradiated to form a stronger crosslinked polymer that is locked in the pores of the substrate. In a specific example, the present investigators blended 50% by weight PVA and 50% by weight PVP powders. The blended powders were then mixed with 50% by weight DMSO in a twin screw mixer at a temperature between 115° C. and 125° C. to a taffy-like consistency. The material was then compression molded into a porous substrate 24 with a bearing 22 projecting from the substrate. The test pieces were subjected to gamma irradiation doses of 50 kGy, 75 kGy, and 100 kGy. The hydrogel was securely fastened to the substrate and presented a lubricious and resilient bearing surface.

Furthermore, if the substrate includes organic substances, such as polymers, irradiating the interface between the hydrogel bearing 22 and the substrate 24 causes the organic groups in the substrate to form covalent bonds with the bearing 22. This crosslinking further enhances their attachment. These covalent bonds are believed to reduce the micromotion between the substrate 24 and bearing material 22 and thus reduce tearing at the interface. For example, any of the above listed hydrogels and mixtures of them will crosslink with organic groups in the substrate when irradiated. In a specific example, the PVA/PVP blend from the previous example was compression molded into a polyethylene foam material and irradiated to crosslink the hydrogel to the foam.

The optimum irradiation dose required to form a secure attachment of the hydrogel within the pores and/or to produce covalent bonding of the hydrogel to a substrate containing organic groups will vary depending on the material. However, doses between 30 kGy and 300 kGy will work for most materials. Doses between 50 kGy and 150 kGy are particularly useful.

If the substrate 24 is metal or ceramic based, the substrate may be surface treated to improve the bond between the bearing material 22 and the substrate 24. For example the surface may be treated by immersing the substrate in nitric acid to clean the surface. In addition, the surface may be treated by immersing the substrate in a mixture of sulfuric acid and hydrogen peroxide to degrade the metal oxides on the surface of the substrate to metal hydroxides. The metal hydroxide containing surfaces may then be treated to bond organic groups to the surface by hydrogen bonding to the metal hydroxides. Such organic groups may be produced by treating the surface with metal alkoxides, organosilanes, hydrocarbon based acids, and/or other suitable materials containing organic groups that will bond to an inorganic surface. Examples of suitable metal alkoxides include titanium di-isopropoxide bif(acetyl acetonate), titanium tri-methacrylate methoxyethoxyethoxide, and/or other suitable metal alkoxides. Examples of organosilanes include hexyltrimethoxysilane, hexamethyldisilazane, and/or other suitable organosilanes. Examples of hydrocarbon based acids include hexanoic acid, octanoic acid, propanoic acid, and/or other suitable hydrocarbon based acids. In a specific example, a tantalum substrate 24 was cleaned in nitric acid, immersed briefly in a mixture of 30 milliliters 30% hydrogen peroxide and 70 milliliters concentrated sulfuric acid, and then coated with hexamethyldisilazane. The PVA/PVP blend from the previous example was compression molded into the pores of the substrate 24 and gamma irradiated with a dose of 50 kGy to create covalent bonding between the hydrogel and the substrate 24.

The hydrogel may advantageously include additives that are released at the surgical site. For example, analgesics and/or antibiotics may be distributed within the water used to hydrate the hydrogel. In use, these additives will migrate out of the hydrogel and into the surrounding tissues to provide localized delivery of the additives to the surgical site. Delivering the additives in the hydrogel bearing 22 may reduce or eliminate the need for systemically administered drugs. A wide variety of analgesics and antibiotics may be used. For example, any of the “-caine” drugs, such as lidocaine, may be used as an analgesic, and any antibiotic, such as tetracycline or gentamicin may be used as an antibiotic.

The substrate 24 is alternatively configured to be anchored directly to tissue or to an optional base plate 50. For anchoring directly to tissue, the substrate may be solid or porous and may be configured to be cemented in place, press-fit in place, or to receive tissue ingrowth. Preferably the substrate 24 includes a second porous region 36 for placement against tissue for receiving tissue ingrowth. For example, a porous tantalum material having a structure similar to that of natural trabecular bone is highly suitable for anchoring to bone. Such a material is described in U.S. Pat. No. 5,282,861 entitled “OPEN CELL TANTALUM STRUCTURES FOR CANCELLOUS BONE IMPLANTS AND CELL AND TISSUE RECEPTORS”. The material is fabricated by vapor depositing tantalum into a porous matrix. The substrate 24 may include protruding pegs or other bone engaging features to further enhance the connection of the substrate to tissue.

The substrate 24 may be formed as an integral part of the bearing 22 by crosslinking the portion that forms the substrate 24 relatively more highly than the portion that forms the bearing surface 26. For example by exposing the substrate side of the bearing 22 to high rate directional irradiation, such as ultraviolet light radiation, the portion closest to the radiation source will form a substrate 24 that is more highly crosslinked than the portion further away from the radiation source that forms the bearing surface 26. Thus an integral substrate 24 is formed having higher strength and rigidity than the bearing surface and suitable for anchoring to tissue or the optional modular base plate 50.

The optional modular base plate 50 includes a body having a substrate 24 engaging portion 52 and an anchor portion 54. The anchor portion 54 may be solid or porous and may be configured to be cemented in place, press-fit in place, or to receive tissue ingrowth. For example, the anchor portion may include a porous metal surface and fixation pegs 56 projecting outwardly to engage a bony anchorage. The modular base plate 50 allows for a variety of bearing 22 and anchor portion 54 configurations to be assembled at the time of surgery to meet a specific patient's needs. The modular base plate 50 further allows for an implant that can be separated into two smaller pieces that are separately passed through a relatively small minimally invasive surgical incision and then assembled in situ. The substrate engaging portion 52 includes a portion for receiving the substrate. In the illustrative example, the substrate engaging portion 52 comprises a generally planar surface 58 on which the substrate 24 rests. A locking mechanism locks the substrate 24 and base plate 50 in engagement. In the illustrative example, the locking mechanism is in the form of a male dovetail 60 projecting upwardly from the base plate 50 and a female dovetail 38 formed in the bottom of the substrate 24 and a peripheral side rail 62 projecting upwardly from the base plate 50. The substrate 24 is assembled to the base plate 50 by slidingly engaging the female dovetail 38 of the substrate 24 with the male dovetail 60 of the base plate 50 and snapping the substrate 24 within the side rail 62.

The bearing surface 26 may receive the opposing natural joint surfaces or the opposing natural joint surfaces may be resurfaced with a prosthetic implant such as optional femoral implant 80 which may be implanted to articulate with the bearing surface 26.

Although examples of a hydrogel implant and its use have been described and illustrated in detail, it is to be understood that the same is intended by way of illustration and example only and is not to be taken by way of limitation. The invention has been illustrated in the context of a tibial articular implant. However, the hydrogel implant may be configured in other shapes and for use at other locations within a patient's body. Accordingly, variations in and modifications to the hydrogel implant and its use will be apparent to those of ordinary skill in the art, and the following claims are intended to cover all such modifications and equivalents.

What is claimed is:

1. An implant for replacing a portion of a skeletal joint, the implant comprising:
a porous substrate; and
an articular surface component comprising a hydrogel attached to the substrate by interdigitation of a portion of the hydrogel into some of the pores of the substrate, the articular surface component defining a bearing surface.

2. The implant of claim 1 wherein the hydrogel comprises a crosslinked hydrogel blend.

3. The implant of claim 2 wherein the hydrogel blend comprises PVA and PVP.

4. The implant of claim 1 wherein the substrate comprises an open cell porous structure having a first porous surface interdigitated with the hydrogel and a second porous surface for receiving tissue ingrowth to anchor the implant adjacent the joint.

5. The implant of claim 4 wherein the substrate comprises a porous metal.

6. The implant of claim 5 wherein the substrate comprises an open cell porous tantalum material.

7. The implant of claim 4 wherein the substrate comprises a porous polymer.

8. The implant of claim 7 wherein the porous polymer comprises foamed polyethylene.

9. The implant of claim 1 wherein the interdigitated portion of the hydrogel is more highly crosslinked than the bearing surface

10. The implant of claim 1 wherein the hydrogel and substrate form covalent bonds between them.

11. The implant of claim 1 wherein the substrate includes a coating forming hydrogen bonds with the substrate and covalent bonds with the hydrogel.

12. The implant of claim 1 wherein the hydrogel includes one or more pharmacological additives.

13. The implant of claim 12 wherein the one or more additives comprise one or more additives selected from the group consisting of analgesics, antibiotics, and growth factors.

14. The implant of claim 1 further comprising:
a modular base plate, the substrate having an engagement surface engageable with the base plate; and
a locking mechanism for locking the substrate in engagement with the base plate.

15. The implant of claim 1 wherein the articular surface is in the form of a tibial aricular surface, the implant further comprising:
a femoral implant engageable with the articular surface in joint articulating arrangement.

16. An implant for replacing a portion of a skeletal joint, the implant comprising:
a substrate having a first portion defining an engagement portion and a second portion,
an articular surface comprising a hydrogel, the hydrogel being attached to the second portion of the substrate,
a modular base plate, the engagement portion of the substrate being engageable with the base plate; and
a locking mechanism for locking the substrate in engagement with the base plate.

17. An implant for replacing a portion of a skeletal joint, the implant comprising:
a hydrogel articular surface; and
an integral substrate for supporting the hydrogel, the substrate being more highly crosslinked than the articular surface.

18. The implant of claim 17 further comprising:
a modular base plate, the substrate having an engagement surface engageable with the base plate; and
a locking mechanism for locking the substrate in engagement with the base plate.

19. A method of forming an implant for replacing a portion of a skeletal joint, the method comprising:
forming an implant having a hydrogel articular surface and a substrate; and
irradiating the implant adjacent to the substrate.

20. The method of claim 19 wherein forming an implant comprises interdigitating a crosslinked hydrogel into a porous substrate and irradiating the implant further crosslinks the hydrogel and locks it in the pores of the substrate.

21. The method of claim 19 wherein the hydrogel articular surface and the substrate comprise separate bodies joined together, the substrate comprising one or more polymers and irradiating the implant adjacent to the substrate causes covalent bonds to form between the hydrogel and the substrate.

22. The method of claim 19 wherein the hydrogel articular surface and the substrate comprise separate bodies joined together, the method further comprising:
treating a portion of the surface of the substrate to enhance the attachment of the hydrogel articular surface to the substrate.

23. The method of claim 22 wherein treating the surface comprises:
attaching organic groups to a portion of the substrate such that irradiating the implant causes covalent bonds to form between the hydrogel articular surface and the organic groups.

24. The method of claim 23 wherein treating the surface comprises:
degrading metal oxides on the surface to form metal hydroxides; and
bonding organic groups to the surface by forming hydrogen bonds between the organic groups and the metal hydroxides.

25. The method of claim 19 wherein the hydrogel articular surface and the substrate are both portions of a unitary continuous body and irradiating the implant comprises exposing the substrate to irradiation such that the substrate becomes more highly crosslinked than the articular surface and forms an integral, more highly crosslinked, substrate of the implant.

26. A method of delivering pharmacological substances to a joint implantation surgical site, the method comprising:
forming a joint implant comprising a hydrogel;
hydrating the hydrogel with a solution containing the pharmacological substance; and
implanting the implant in joint articulating arrangement within a skeletal joint such that as the implant articulates within the joint, the pharmacological substance is released.
